(19) United States
(12) Patent Application Publication
(10) Pub. No.: US 2026/0108200 A1
(43) Pub. Date: Apr. 23, 2026

(54) TIME DOMAIN METHODS AND SYSTEMS TO IDENTIFY HARMONIC VERSUS RANDOM ACTIVITY WIHTIN SPECIFIC FREQUENCY RANGES OF EEG OUTPUT

(71) Applicants: Hyundai Motor Company, Seoul (KR); Kia Corporation, Seoul (KR)

(72) Inventor: Christopher Scheib, Nicholasville, KY (US)

(73) Assignees: Hyundai Motor Company, Seoul (KR); Kia Corporation, Seoul (KR)

(21) Appl. No.: 19/363,183

(22) Filed: Oct. 20, 2025

Related U.S. Application Data
(60) Provisional application No. 63/709,464, filed on Oct. 20, 2024.

(51) Int. Cl. A61B 5/374; A61B 5/00

(52) U.S. Cl. CPC A61B 5/374; A61B 5/725; A61B 5/743

(57) ABSTRACT

Systems and methods for EEG signal analysis configured to identify random and harmonic segments of an EEG signal associated with brain activity of a subject, and determine and display at least one or both of: a filtered EEG signal from the EEG signal, wherein the identified random and harmonic segments of the EEG signal have been which have been removed and the remaining portion of the EEG signal is displayed, and/or a non-filtered or partially filtered EEG signal from the EEG signal wherein the identified random and harmonic segments are indicated or identified in the display.

### CROSS REFERENCE TO RELATED APPLICATION

This application claims priority to U.S. Provisional Application No. 63/709,464 filed Oct. 20, 2024, the disclosure of which is incorporated herein.

### BACKGROUND OF THE INVENTION

### Field of the Invention

The present invention relates to tracking and assessing changes in detected signals generated by an electroencephalogram (EEG) to identify certain conditions that are correlated with the anesthetic state of a subject.

The EEG is utilized to assess and track changes in the anesthetic state. Many methods have been used to process the EEG to aid in this endeavor. Several theories of the link between components of the EEG signal and the neuroscience of the anesthetic state have been proposed. One theory is that there are oscillations consisting of several cycles which occur during the anesthetic state which are the result of the effects of anesthetic agents and the effects of the nervous systems processing surgical stimulation during anesthesia. Identifying these oscillations in the signal would inform the anesthesia provider of details of the anesthetic state in near real time.

One example of these oscillations occurs in the alpha frequency range (about 7 Hertz to about 14 Hertz) in the front of the cortex. The neuroscience mechanism is thought to be like an oscillation in natural sleep called the “spindle wave.” The spindle process produces sets of oscillations where every cycle is nearly identical in cycle length. These oscillations will be termed “harmonic.” There are oscillations in this frequency range in the EEG that are not related to the “spindle” process and do not have sets of identical cycles. These will be termed “random.” In the alpha range there is “alpha harmonic’ and “alpha random” activity. There are four other frequency bands that are commonly used; from low frequency to high they are delta, theta, alpha, beta, and gamma. While the spectrum of the EEG could be divided differently, it is traditionally divided using the aforementioned scheme.

As a time-varying signal, EEG can be viewed, analyzed, and interpreted in two distinct ways, or domains. The common way of viewing EEG data is in the time domain, with time plotted on the x axis, and potential (voltage) on the y axis. Alternatively, EEG can be viewed in the frequency domain. The frequency domain method of identifying and quantifying this part of the EEG signal usually involves the Fourier method to create an EEG spectrum and subtract a baseline from a peak in the alpha range. This approach is used by electrical engineers in the field of signal processing for signals generated by processes that are different from the processes that generate the EEG. However, the frequency domain methods do not identify sequences of oscillations with the same cycle length.

There are important applications of EEG analysis which have not been possible or have been extremely inconvenient with prior art EEG systems. One important application is in the monitoring of cerebral functions, namely brain activity, while the subject is in an unconscious or anesthetic state.

The “raw” EEG has activity from many sources within the brain with frequencies varying from greater than 0 to about 45 hertz. The brain has oscillations of several cycles at multiple narrow frequencies. These can be called “harmonic.” The rest of the signal is called random. The Fourier method can be used to find harmonic activity in the delta and alpha frequency bands. A peak minus a drawn baseline of a spectra has been called harmonic with the baseline called random or noise.

Accordingly, there is a need for the systems and methods of the invention which, among other things, resolves the issues mentioned herein, separates the EEG signal into frequency ranges using modifiers or filters, identifies and quantifies the part of the EEG signal which produces such repetitive cycles.

### SUMMARY OF THE INVENTION

The invention is directed to addressing the deficiencies in the art.

In some embodiments, the invention is directed to an EEG signal analysis system and method which is configured to identify random and harmonic segments of an EEG signal associated with brain activity of a subject, and determine and display, on a real-time basis, at least one or both of: a filtered EEG signal from the EEG signal, wherein the identified random and harmonic segments of the EEG signal which have been removed and the remaining portion of the EEG signal is displayed, and/or a non-filtered or partially filtered EEG signal from the EEG signal wherein the identified random and harmonic segments are indicated or identified in the display.

In some embodiments, the invention is directed to an EEG signal analysis system and method which identifies random or harmonic segments in an EEG signal associated with brain activity of a subject, and generates from the EEG signal, on a real-time basis, a display or illustration of the EEG signal wherein the amount of harmonic or random activity identified in the EEG signal is displayed in or with various signal terms, parameters or characteristics, such as amplitude, power, and duration, being indicated, and wherein the display may further include an illustration of the relationships between the identified random or harmonic segments in the EEG signal based on such signal terms, parameters or characteristics.

In some embodiments, the EEG signals are received from multiple channels, prior to being sampled, digitized and stored in a memory or database during a continuing sequence of intervals of predetermined time duration. Digitized waveforms may be generated and stored for each channel.

Some embodiments of the invention are directed to systems and methods of the invention are configured to separate the EEG signal into frequency ranges with filters, among other things. This is referred to herein as a “time domain” method.

In some embodiments of the invention, each of the filtered signal streams is evaluated with any of several known methods to identify segments with sequences of cycles with nearly identical cycle length. Those segments (harmonic) and the remaining segments (random) can be used to create an indication of the anesthetic state. One example is used in the illustrations below where the amount of power in the harmonic and random segments is calculated and graphed versus time or versus agent concentration.

In some embodiments, the identified segments are also be used to create a filter which splits the signal in the specified frequency range into two outputs. The harmonic segments and the random segments could be displayed separately or identified within one output. The output of this filter could be displayed enabling the practitioner to visualize relevant changes in the EEG. A change in the amount of random activity can be as important as a change in the amount of harmonic oscillations.

Systems and methods of the invention have been tested against the spectral method. In some examples the two methods produced very similar results. This is evidence for the validity of the systems and methods of the invention. There are also examples were the new method showed a decline in the amount of harmonic activity while the spectra showed a large peak over baseline and vice versa. The clinical circumstances of those examples indicate that the systems and methods of the invention provide an earlier warning of an anesthetic state that was close to the edge of changing while the spectra method indicated that all was well.

In some embodiments, the systems and methods of the invention are able to identify similar multicycle oscillations in the other frequency bands of the EEG. In many cases there is no peak in the frequency range where these oscillations occur. It is possible that oscillations other than alpha oscillations occur. The field of neuroscience has described oscillations in the other frequency ranges. Bicoherence and bispectral analysis are methods which have identified phase relationships between oscillations at multiple frequencies during anesthesia.

For example, some embodiments of the invention are directed to a system for analyzing EEG signals, comprising an electrode array, a processing device, a display and a memory, wherein the memory contains executable programming configured to: process via the processing device, an EEG signal received from the electrode array, the EEG signal being associated with brain activity of a subject, wherein the processing device identifies one or more random segments and one or more harmonic segments of the EEG signal in at least one frequency band of the plurality of frequency bands; and cause to display by the processing device, on a real-time basis, at least one or both of: a filtered EEG signal from the EEG signal, wherein the filtered EEG signal comprises a display of the EEG signal with the one or more random and harmonic segments of the EEG signal removed; and a modified EEG signal, wherein the modified signal includes a display of the EEG signal with the one or more random and one or more harmonic segments visually identified.

In some embodiments, the systems and methods of the invention are further configured to display of a visually identifiable comparison between the one or more random segments and the one or more harmonic segments. The display of the visually identifiable comparison may be within the same or a different frequency band of the one or more frequency bands.

In some embodiments, the systems and methods of the invention are further configured to display a visually identifiable comparison between the one or more harmonic segments of a first frequency band of the one or more frequency bands and a second frequency band of the one or more frequency bands.

In some embodiments, the systems and methods of the invention are further configured to display of a visually identifiable comparison between the one or more random segments of a first frequency band of the one or more frequency bands and a second frequency band of the one or more frequency bands.

The visually identifiable comparisons may be conducted over one or more periods of time.

In some embodiments, the EEG signal comprises multiple channels and the EEG signal may be divided into time intervals. In some embodiments, the EEG signal is received over a period of time.

In some embodiments, the processing device is further configured to: identify in the EEG signal a first amount of harmonic activity identified in the EEG signal and a second amount of random activity identified in the EEG signal, the first amount of harmonic activity and the second amount of random activity being identified over one or more periods of time; generate for display on the display device one or more characteristics of the EEG signal for each of the one or more periods of time. The one or more characteristics may comprise amplitude, power and duration of the identified harmonic or random oscillation for the one or more periods of time.

In some embodiment, the system is further configured to generate a display of one or more relationships between the first amount of harmonic activity and the second amount of random activity identified in the EEG signal based on the one or more characteristics. The amount of power in the first amount of harmonic activity and the second amount of random activity may be calculated and displayed as a graph of power versus time and power versus an agent concentration.

In some embodiments, the processing device is configured to separate the EEG signal into frequency ranges comprising a plurality of filtered signal streams.

In some embodiments, the system is further configured to identify the one or more harmonic segments having one or more sequences of cycles having substantially identical cycle length in each of the plurality of filtered signal streams.

In some embodiments, the processing device is further configured to generate a filter based on the one or more harmonic segments and the one or more random segments, wherein the processing device divides the signal in a specified frequency range into two outputs.

In some embodiments, the one or more harmonic segments and the one or more random segments are displayed separately on the display device.

Some embodiments of the invention are directed to a method of analyzing EEG signals using an electrode array, a processing device, a display and a memory, wherein the memory contains executable programming, the method comprising the steps of: processing via the processing device, an EEG signal received from the electrode array, the EEG signal being associated with brain activity of a subject, wherein the processing device identifies one or more random segments and one or more harmonic segments of the EEG signal in at least one frequency band of the plurality of frequency bands; and causing to display by the processing device, on a real-time basis, at least one or both of: a filtered EEG signal from the EEG signal, wherein the filtered EEG signal comprises a display of the EEG signal with the one or more random and harmonic segments of the EEG signal removed; and a modified EEG signal, wherein the modified signal includes a display of the EEG signal with the one or more random and one or more harmonic segments visually identified.

In some embodiments, the aforementioned method further comprises the steps of: identifying in the EEG signal a first amount of harmonic activity identified in the EEG signal and a second amount of random activity identified in the EEG signal, the first amount of harmonic activity and the second amount of random activity being identified over one or more periods of time; and generating for display on the display device one or more characteristics of the EEG signal for each of the one or more periods of time.

In some embodiments, the aforementioned method further comprises the step of generating a display of one or more relationships between the first amount of harmonic activity and the second amount of random activity identified in the EEG signal based on the one or more characteristics.

In some embodiments, the aforementioned method further comprises the step of identifying the one or more harmonic segments having one or more sequences of cycles having substantially identical cycle length in each of the plurality of filtered signal streams.

In some embodiments, the aforementioned method further comprises the step of generating a filter based on the one or more harmonic segments and the one or more random segments, wherein the processing device divides the signal in a specified frequency range into two outputs.

In some embodiments, the aforementioned method further comprises the step of displaying the one or more harmonic segments and the one or more random segments on the display device.

Other features of embodiments of the present disclosure will be apparent from accompanying drawings and from the detailed description that follows.

### BRIEF DESCRIPTION OF THE DRAWINGS

FIG. 1 is an electrical block diagram of an exemplary embodiment of a real-time EEG analyzer constructed in accordance with the invention and configured to operate the methods of the invention; and

FIGS. 2-21 illustrate various sample displays and printed output provided by an analyzer configured to operate the methods of the invention, such as the analyzer depicted in FIG. 1.

### DETAILED DESCRIPTION OF SOME EMBODIMENTS OF THE INVENTION

Systems and methods are disclosed for employing a plurality of methods and algorithms to enable analysis of EEG signals as described herein, and the development of and/or integration of such methods and algorithms in analyzer systems.

Various methods described herein may be practiced by combining one or more machine-readable storage media containing the code according to the invention with appropriate standard computer hardware to execute the code contained therein. An apparatus for practicing various embodiments of the invention may involve specialized hardware components and one or more computers (or one or more processors within a single computer), and storage systems containing or having network access to computer program(s) coded with the methods of the invention being accomplished by modules, routines, subroutines, or subparts of a computer program product.

This invention may be embodied in many different forms than described herein and should not be construed as limited to the embodiments set forth herein. These embodiments are provided so that this disclosure will be thorough and complete and will fully convey the scope of the invention to those of ordinary skill in the art. Moreover, all statements herein reciting embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure).

Thus, for example, it will be appreciated by those of ordinary skill in the art that the figures represent exemplary apparatus and results to illustrate systems and methods which embody the teachings of this invention. The functions of the various elements shown in the figures may be provided through the use of dedicated hardware as well as hardware capable of executing associated software. Similarly, any components shown in the figures are conceptual only. Their function may be carried out through the operation of similar components, program logic, through dedicated logic, through the interaction of program control and dedicated logic, the particular technique being selectable by the entity implementing this invention.

Specific details are given in the following description to provide a thorough understanding of the embodiments. However, it will be understood by one of ordinary skill in the art that the embodiments may be practiced without these specific details. For example, circuits, systems, processes, and other components may be shown as components in block diagram form, combined or separated, in order not to obscure the embodiments in unnecessary detail. In other instances, well-known input devices, output devices, circuits, amplifiers, interfaces, communication devices, processes, algorithms, structures, and techniques may be left out or shown without unnecessary detail in order to avoid obscuring the exemplary embodiments of the invention.

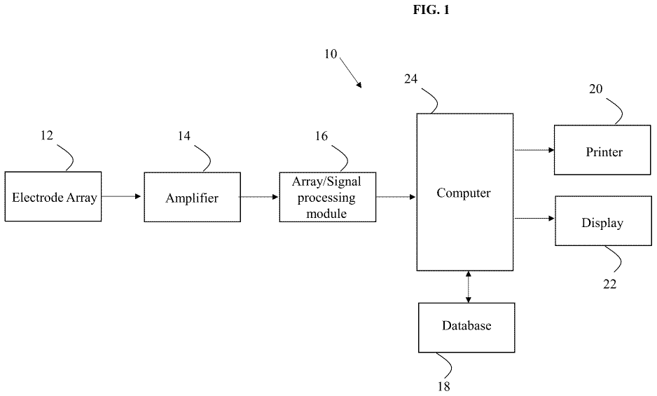

FIG. 1 shows an exemplary EEG signal analyzer 10 that is constructed in accordance with, and configured to deliver, the systems and methods of the invention. Analyzer 10 includes an EEG electrode array 12, EEG multi-channel amplifier circuit 14, multi-channel array and EEG signal processing module 16, memory or database 18, printer (or other hard copy device) 20, display 22 and a computer 24, which may also function as a control device for system 10. Computer 24 may also include or be in communication with a server.

System 10 is configured to record and analyze EEG signals received from EEG electrode array 12 and provide an output on a real-time basis through display 22 or printer 20 which indicates the frequency response to EEG signals from various channels in each of a plurality of frequency bands of interest, such as the five frequency bands (delta, theta, alpha, beta and gamma).

EEG electrode array 12 generally includes a plurality of EEG electrodes which are placed in contact with the scalp of a human subject, typically at various sites on the subject's head, and may also include one or more reference electrode sites. The EEG has multiple channels with each channel representing one of the EEG electrode sites of array 12. EEG multichannel amplifier circuit 14 includes a differential amplifier for each channel, which amplifies the potential difference between a reference potential and the potential at the electrode site for that particular channel. The output of EEG multichannel amplifier circuit 14 may be an analog EEG signal for each channel.

Array and EEG signal processing module 16 receives the analog EEG signals from EEG multichannel amplifier circuit 14. Array and signal processing module 16 samples the analog EEG signal for each channel. The sampled analog values for each channel may be converted to digital values, and are stored by signal processing module 16 in database 18. The sampling, digitizing and storing occurs over a continuing series of time intervals. In some embodiments, the lowest frequency of interest is one Hertz, and therefore each interval has a duration of at least one second. The stored digital sample values for each channel represent the amplitude of the EEG signal as a function of time (i.e. a digitized waveform) during that interval.

In operation, EEG signals processed by signal processing module 16 are analyzed in real-time by computer 24 to determine and identify random and harmonic segments of the EEG signal, and the results thereof may either be printed via printer 20 or displayed on display 22. For example, a display generated by computer 24 may include one or both of a filtered EEG signal associated with brain activity of a subject in which the identified random and harmonic sections of EEG signal have been removed and/or a non-filtered or partially filtered EEG signal in which the identified random and harmonic sections are also identified on the display 22.

Computer 24 of system 10 may also, in real-time, identify random or harmonic segments in the EEG signal associated with brain activity of a subject, and generate from the EEG signal a display on display 22 of the amount of harmonic or random activity identified in the EEG signal by determining various signal characteristics, such as the amplitude, power, and duration of the EEG signal, and thereafter display on display 22 an illustration of the relationships between the identified random or harmonic segments in the EEG signal based on such signal characteristics.

It is envisioned that embodiments of the invention may be used to assist medical personnel in various ways. For example, the invention may be used to produce numerical results or parameters which identify and notify or indicate (either audible, visual, or both) the amount of harmonic or random activity in at least one or more various terms, such as amplitude, power and duration, which further indicates the relationships between identified segments in at least one or more of the various terms, such as amplitude, power, timing and correlation. Embodiments of the invention may also be used to display the EEG signal that remains after removing identified random or harmonic segments. This can be done with either a display of the remaining signal after being filtered or indicating identified random and harmonic sections in a display of the raw or partially processed or filtered EEG signal.

In addition to the foregoing, embodiments of the invention, such as the systems and methods disclosed herein, may be configured to filter the EEG signal into several frequency bands (such as, but not limited to, delta, theta, alpha, beta, and gamma,) and identify the oscillations, such as through a method that can identify oscillations with cycles of similar frequency, which may be within the range of about 90 to about 100%; the same or substantially the same oscillations of similar frequency in the range of about 95 to about 100%; the same, or substantially identical oscillations of similar frequency, such as in the range of about 98-100%. The identified segments can be used in accordance with the invention in several ways. For example, signal characteristics and parameters can be identified, such as for example, the harmonic or random power in each of five standard frequency bands, thus enabling the invention to identify ten segment parameters associated with the EEG signal. In addition to power (and related concepts like amplitude) the length of the set of oscillations can be an identified parameter, and relationships between the ten identified segment parameters, such as timing and the identification of oscillations can also be determined. Since there are multiple channels of the EEG signal, relationships between a parameter from one channel and the same or different parameter from another channel may be useful. In some embodiments, cycles which are within a certain degree of similarity, such as within the ranges discussed above, may be identified and visually distinguished or removed from the EEG signal.

In addition to the parameter approach described above, embodiments of the invention can also be configured to display a modified EEG signal approach. For example, the filtered bands can be displayed separately with each of the harmonic and random sections identified (that is, five windows); the whole EEG can be displayed with harmonic (or random) areas identified (one window) by coloring or otherwise visually distinguishing the EEG tracing or by markings on the background. As an illustration, the alpha harmonic may be displayed with the “raw” EEG with alpha harmonic oscillation brightly colored so the oscillations are easily identifiable, thus allowing for example, a practitioner to make adjustments to the anesthetic. Oscillations in other frequencies may also be indicated simultaneously on the one window display or on multiple windows. In some embodiments, an analyzer of the invention may further include a virtual or physical actuator or switch which is configured to turn the visual indications on and off separately.

It should be understood that the oscillations may also be identified and visually distinguished either with or without filtering the EEG signal. For example, the analyzer may apply a plurality of templates for comparison to the raw EEG signal to identify a match between a template and the signal.

It should also be understood that the systems and methods set forth herein may be advantageously used to analyze brain activity responsive to an anesthetic state, but is not limited thereto, and can be used to analyze brain activity for subjects in other states, such as unconscious and conscious states including natural sleep and coma.

The following figures demonstrate samples of analyzer output and results, along with other features and benefits provided by exemplary systems and methods of the invention, such as an analyzer 10.

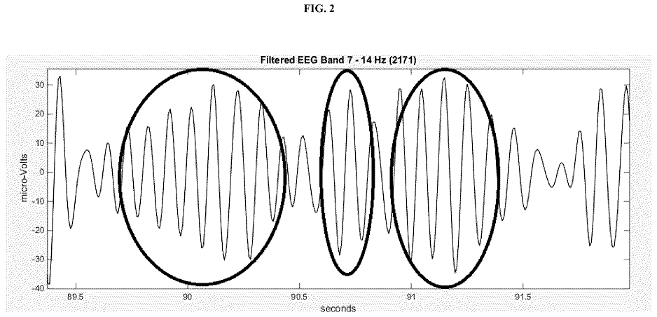

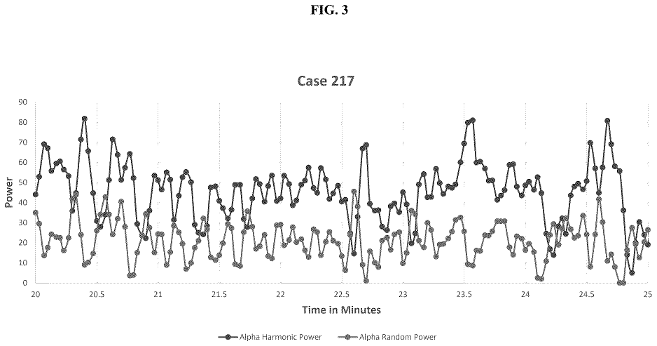

FIG. 2 illustrates about 2.5 seconds of the output of a 7-14 Hz pass filter from an EEG during anesthesia. The circles indicate what the method may identify as harmonic. FIG. 3 provides a graph below shows values for alpha harmonic and random power in 2 second intervals.

Analysis of the harmonic oscillations, the random oscillations, and the relationships between the various oscillations, add information about the anesthetic state which may help the anesthesia practitioner make appropriate clinical decisions. Examples of how this method of EEG analysis indicates changes in the anesthetic state follow. In these examples the graphs indicate one minute average values for the parameters

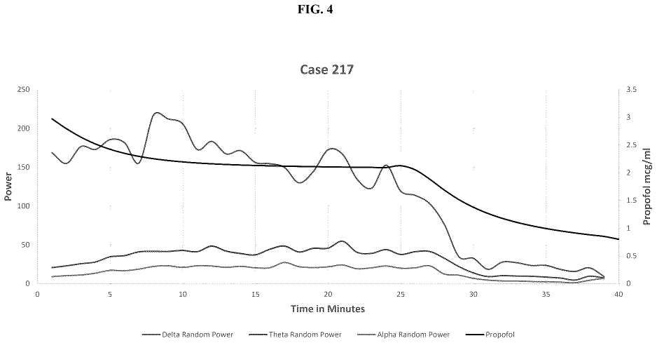

FIG. 4 illustrates that the random power components decrease (not increase) in power in the order of declining frequency ranges, that is, Delta>Theta>Alpha . . . over a range of anesthetic agent concentration.

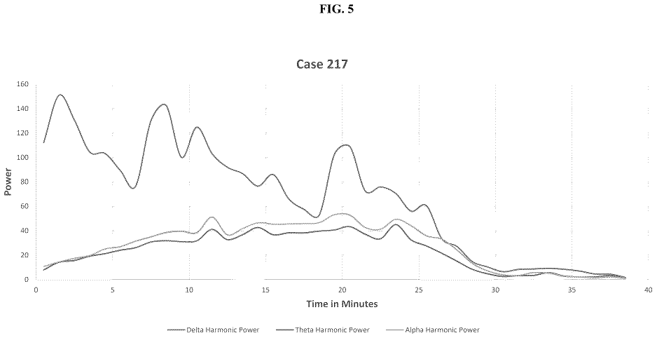

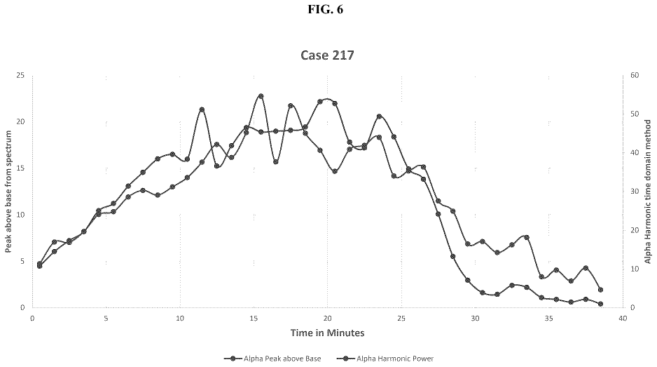

As shown in FIG. 5 particularly, when the alpha harmonic component has more power than the lower frequency theta harmonic component, it indicates a particular level of anesthesia. Some practitioners look at the spectrogram for more power in alpha than theta and adjust agent concentration to achieve this. (An alpha peak higher than a theta trough.) The graph shown in FIG. 6 illustrates that the time domain and the frequency domains produced similar results.

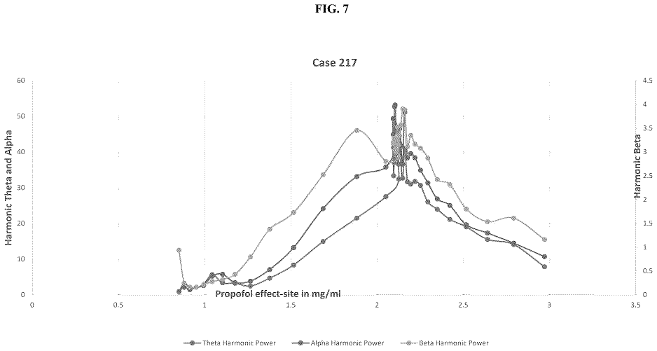

The graph in FIG. 7 shows that the harmonic components can have a maximum value in a narrow range of anesthetic agent concentration. This phenomenon can be used to indicate the level of anesthetic effect. This case had enough opioid analgesia to prevent surgical stimulation from affecting the harmonic components very much. The variability in this case is likely due to mechanisms that the nervous system uses to maintain a consistent neuron membrane voltage. Like a thermostat, the feedback mechanism oscillates between a higher and a lower value.

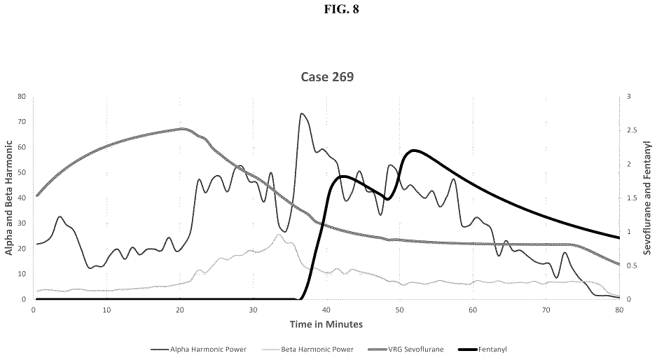

The graph in FIG. 8 shows a different case. Notice what happened between 20 and 40 minutes. A declining sevoflurane (inhaled anesthetic agent) resulted in a gradual increase in the beta harmonic and then a sudden decrease in the alpha harmonic at 33 minutes. Adding fentanyl (intravenous opioid analgesic) resulted in a rapid increase in the alpha harmonic and a rapid decrease in the beta harmonic. (The timing of the documentation of the fentanyl is apparently about two minutes late.)

This comparison of the beta harmonic with the alpha harmonic can be used to assess the effectiveness of the analgesic component of the anesthetic. In some embodiments, the analyzer of the invention, such as analyzer 10, is configured to compare harmonic segments to other harmonic segments (such as other harmonic segments in different frequency bands) over a period of time to track changes. As demonstrated herein, there may be a concentration of anesthetic where there is a maximum amount of harmonic activity in a frequency range that is different for each of the frequency ranges. This maximum activity is typically surrounded by declining activity as the anesthetic agent concentration rises or falls.

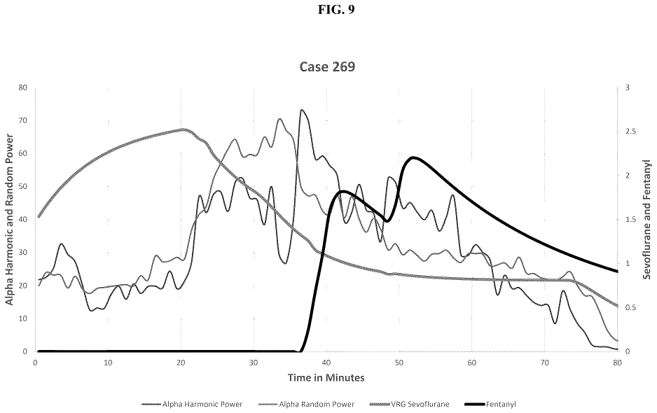

The graph in FIG. 9 shows that as the sevoflurane was decreased the alpha random exceeded the alpha harmonic component until fentanyl administration reversed the relationship.

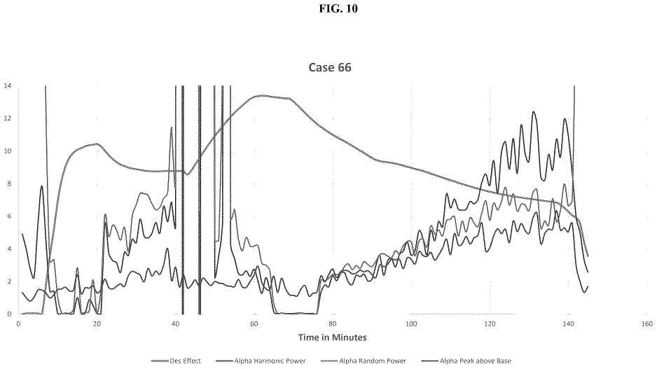

The case in the graph of FIG. 10 shows that as the desflurane (inhaled agent) was decreased (without any opioid analgesia administered) The alpha harmonic increased more than the alpha random or the peak above baseline.

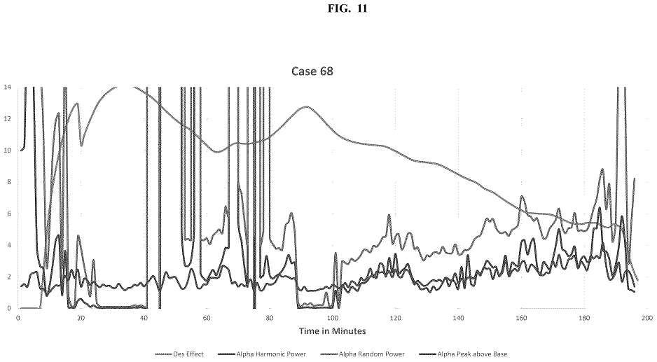

The case in the graph of FIG. 11 shows that as the desflurane was decreased (without any opioid analgesia administered) The alpha random component exceeded the alpha harmonic component and the peak above baseline had a very low value.

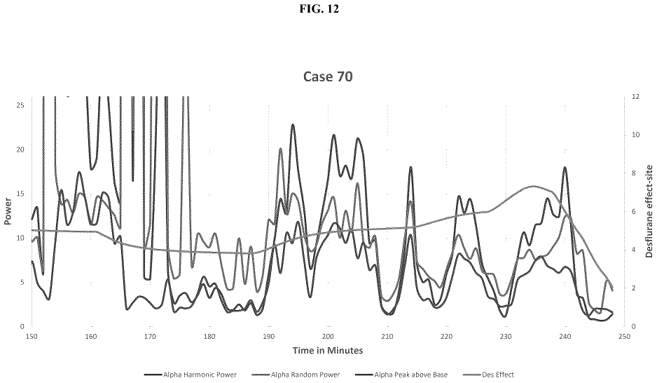

The case shown in FIG. 12 also had no analgesic component. There were several EEG responses which were likely due to the nervous system responding to surgical stimulation. The desflurane concentration was high enough to prevent awareness. If the patient had become aware, there was not enough paralytic agent to prevent movement.

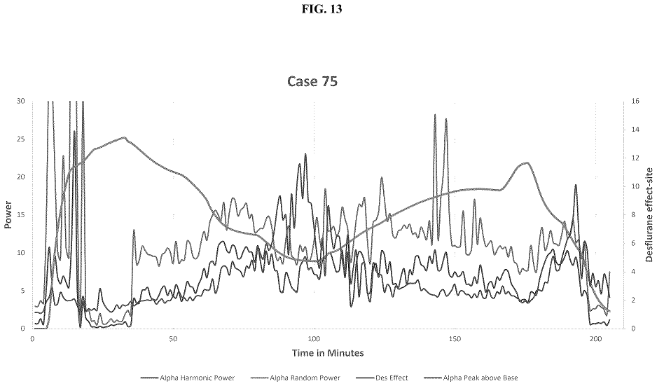

The case shown in FIG. 13 shows a large alpha peak above baseline at a low concentration of desflurane with a low level of the alpha harmonic component. One usefulness of the time domain method is to verify the alpha peak in a spectrogram.

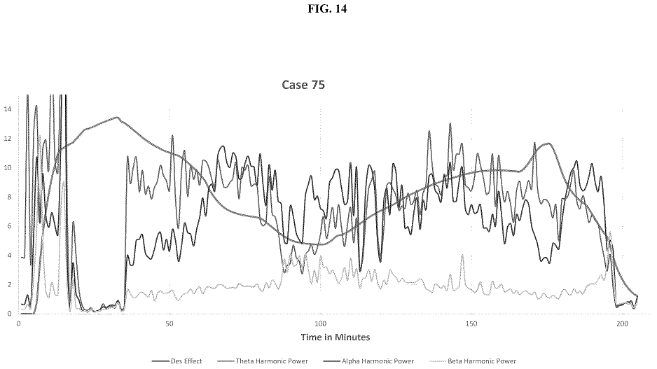

The graph shown in FIG. 14 (same case as shown in FIG. 13) illustrates that the relative amounts of theta, alpha, and beta harmonic components change with changes in the anesthetic concentration. Delta is not shown in this graph but it is also useful to assess the “effective” concentration of the anesthetic agent.

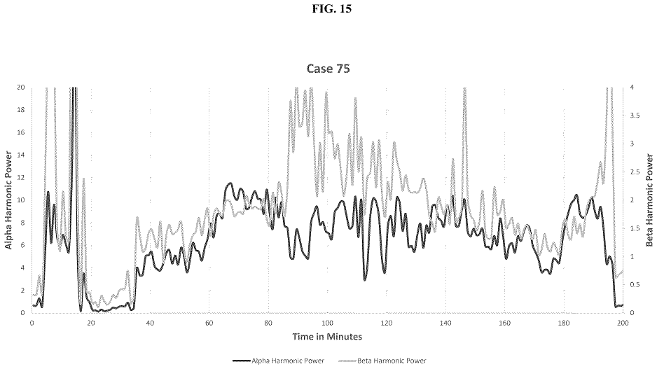

It should be noted that, as shown in FIG. 15, at the low concentration of the anesthetic agent the beta harmonic changes opposite of the alpha (anticorrelation) and at higher concentrations they correlate. This is another clue of a change in the anesthetic state.

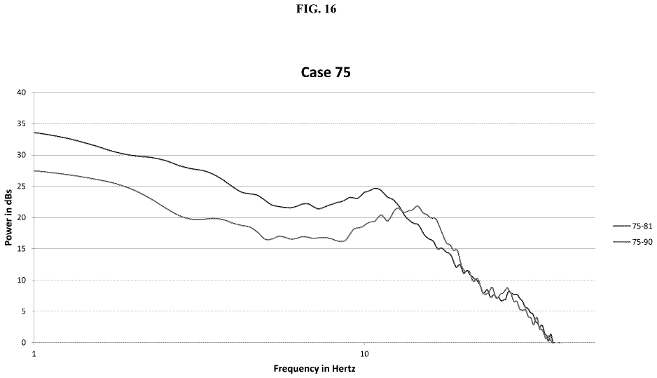

The graph shown in FIG. 16 is the log-log spectra of the same case at minutes 81 and 90. It is consistent with the time domain method.

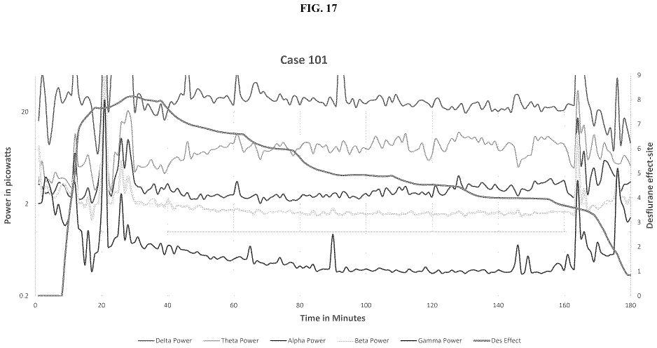

The case shown in FIG. 17 is an example where the alpha never exceeded the theta. The method used in the graph below is the traditional RMS and does not distinguish between harmonic and random.

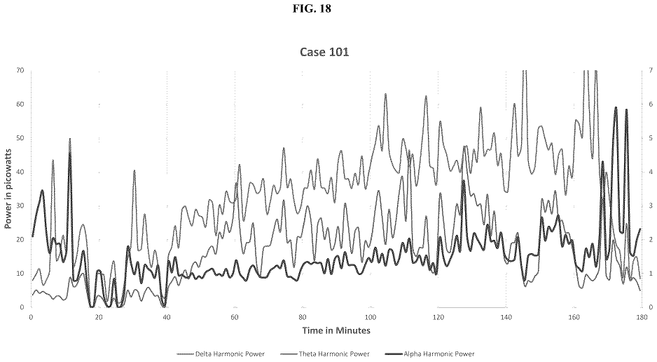

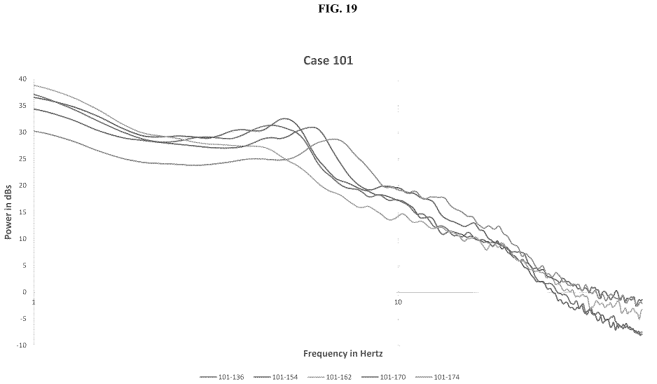

The theta harmonic exceeded the alpha harmonic until the last 10 minutes when the desflurane was very low as shown in FIG. 18. As shown in FIG. 19, the spectra from the same case of FIG. 18 show the peak to be in the theta range.

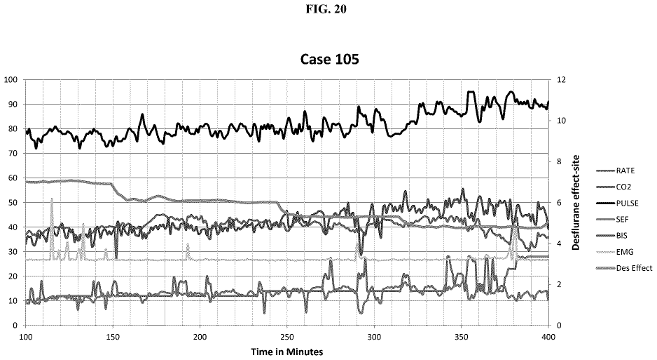

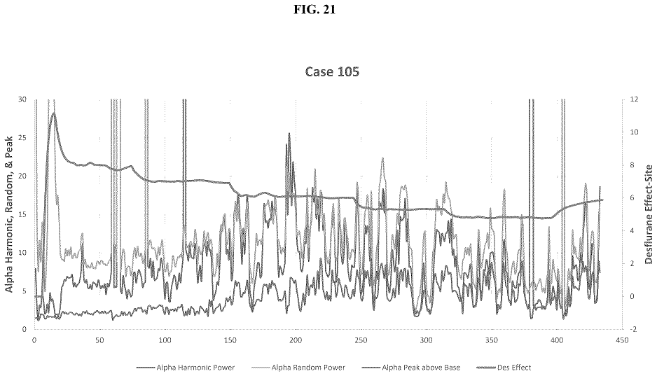

As shown in FIG. 20, pulse rate and respiratory rate rise with surgical stimulation and EEG indicators react as if anesthetic agent increased. As shown in FIG. 21, the three alpha band indicators move together with multiple paradoxical responses. This case illustrates the “paradoxical” arousal.

In some embodiments, the system and method of the invention as described herein are implemented by a computer program configured to provide a computer-readable medium operable on a computerized platform and configured to communicate with remote devices.

In some embodiments the invention is implemented by a computer system includes processing resources, a main memory, a read-only memory (ROM), a data storage device, and a communication interface. The computer system includes at least one processor for processing information stored in the main memory, such as provided by a random access memory (RAM) or other dynamic storage device, for storing information and instructions which are executable by the processor. The main memory also may be used for storing temporary variables or other intermediate information during execution of instructions to be executed by the processor.

The communication interface enables the computer system to communicate with one or more networks (e.g., cellular network) through use of the network link (wireless or a wire). Using the network link, the computer system can communicate with one or more computing devices, and one or more servers. The processor is configured with software and/or other logic to perform any one or more processes, steps and other functions described herein.

Examples described herein are related to the use of the computer system for implementing the techniques described herein. According to one embodiment, those techniques are performed by the computer system in response to the processor executing one or more sequences of one or more instructions contained in the main memory (e.g., applying a filter to EEG output, identifying certain conditions, and notifying of identified conditions). In alternative implementations, hard-wired circuitry may be used in place of or in combination with software instructions to implement examples described herein. Thus, the examples described are not limited to any specific combination of hardware circuitry and software.

In the embodiments described herein the invention may be configured to receive input data relating to EEG results from a subject, apply a filter and produce filtered results.

It should be understood that the methods, techniques, and actions performed by a computing device as described herein are performed programmatically, or as a computer-implemented method. Programmatically, as used herein, means through the use of code or computer-executable instructions. These instructions can be stored in one or more memory resources of the computing device. A programmatically performed step may or may not be automatic. The exemplary embodiment can be implemented using programmatic modules, engines, or components. A programmatic module, engine, or component can include a program, a sub-routine, a portion of a program, or a software component or a hardware component capable of performing one or more stated tasks or functions. As used herein, a module or component can exist on a hardware component independently of other modules or components. Alternatively, a module or component can be a shared element or process of other modules, programs or machines.

The exemplary embodiments described herein may be implemented, in whole or in part, on computing devices such as servers, desktop computers, cellular or smartphones, personal digital assistants (e.g., PDAs), laptop computers, printers, digital picture frames, network equipment (e.g., routers) and tablet devices. Memory, processing, and network resources may all be used in connection with the establishment, use, or performance of any embodiment described herein (including with the performance of any method or with the implementation of any system).

One or more embodiments described herein may be implemented through the use of instructions that are executable by one or more processors. These instructions may be carried on a computer-readable medium. Machines shown or described with figures below provide examples of processing resources and computer-readable mediums on which instructions for implementing embodiments of the invention can be carried and/or executed. In particular, the numerous machines shown with embodiments of the invention include processor(s) and various forms of memory for holding data and instructions. Examples of computer-readable mediums include permanent memory storage devices, such as hard drives on personal computers or servers. Other examples of computer storage mediums include portable storage units, such as CD or DVD units, flash memory (such as carried on smartphones, multifunctional devices or tablets), and magnetic memory. Computers, terminals, network enabled devices (e.g., mobile devices, such as cell phones) are all examples of machines and devices that utilize processors, memory, and instructions stored on computer-readable mediums. Additionally, embodiments may be implemented in the form of computer-programs, or a computer usable carrier medium capable of carrying such a program.

In some embodiments, the methods, systems, and media disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages. The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft^{®}.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft^{®} SQL Server, mySQL™, and Oracle^{®}. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash^{®} Actionscript, Javascript, or Silverlight^{®}. In some embodiments, a web application is written to some extent in a server-side coding language such as Active Server Pages (ASP), ColdFusion^{®}, Perl, Java™, JavaServer Pages (JSP), Hypertext Preprocessor (PHP), Python™, Ruby, Tcl, Smalltalk, WebDNA^{®}, or Groovy. In some embodiments, a web application is written to some extent in a database query language such as Structured Query Language (SQL). In some embodiments, a web application integrates enterprise server products such as IBM^{®} Lotus Domino^{®}.

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein. In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C #, Objective-C, Java™, Javascript, Pascal, Object Pascal, Python™, Ruby, VB.NET, WML, and XHTML/HTML with or without CSS, or combinations thereof. Suitable mobile application development environments are available from several sources.

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB. NET, or combinations thereof. Compilation is often performed, at least in part, to create an executable program. In some embodiments, a computer program includes one or more executable complied applications.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The algorithms and displays presented herein are not inherently related to any particular computer or other apparatus. Various general-purpose systems may also be used with programs in accordance with the teachings herein, or it may prove convenient to construct more specialized apparatus to perform the method steps. The structure for a variety of these systems will appear from the description herein. In addition, the embodiments are not described with reference to any particular programming language. It will be appreciated that a variety of programming languages may be used to implement the teachings of the embodiments as described herein, and any references herein to specific languages are provided for the purposes of enablement and best mode. Those skilled in the art will appreciate that the types of software and hardware used are not vital to the full implementation of the methods of the invention.

While exemplary systems and methods, and applications of methods of the invention, have been described herein, it should also be understood that the foregoing is only illustrative of a few particular embodiments with exemplary and/or preferred features, as well as principles of the invention, and that various modifications can be made by those skilled in the art without departing from the scope and spirit of the invention. Therefore, the described embodiments should not be considered as limiting of the scope of the invention in any way. Accordingly, the invention embraces alternatives, modifications and variations which fall within the spirit and scope of the invention as set forth herein, in the claims and equivalents thereto.

1. A system for analyzing EEG signals, comprising an electrode array, a processing device, a display device and a memory, wherein the memory contains executable programming configured to:
a) process via the processing device, an EEG signal received from the electrode array, the EEG signal being associated with brain activity of a subject, wherein the processing device identifies one or more random segments and one or more harmonic segments of the EEG signal in one or more frequency bands; and
b) cause to display by the processing device, on a real-time basis, at least one or both of: a filtered EEG signal from the EEG signal, wherein the filtered EEG signal comprises a display of the EEG signal with the one or more random segments and the one or more harmonic segments of the EEG signal removed; and a modified EEG signal, wherein the modified signal includes a display of the EEG signal with the one or more random segments and the one or more harmonic segments visually identified.

2. The system as recited in claim 1, further comprising a display of a visually identifiable comparison between the one or more random segments and the one or more harmonic segments.

3. The system as recited in claim 2, wherein the display of the visually identifiable comparison is within the same frequency band of the one or more frequency bands.

4. The system as recited in claim 1, further comprising a display of a visually identifiable comparison between the one or more harmonic segments of a first frequency band of the one or more frequency bands and a second frequency band of the one or more frequency bands.

5. The system as recited in claim 4, wherein the visually identifiable comparison is conducted over one or more periods of time.

6. The system as recited in claim 1, further comprising a display of a visually identifiable comparison between the one or more random segments of a first frequency band of the one or more frequency bands and a second frequency band of the one or more frequency bands.

7. The system as recited in claim 1, wherein the processing device is further configured to:
identify in the EEG signal a first amount of harmonic activity identified in the EEG signal and a second amount of random activity identified in the EEG signal in at least one frequency band of the plurality of frequency bands, the first amount of harmonic activity and the second amount of random activity being identified over one or more periods of time; generate for display on the display device one or more characteristics of the EEG signal for each of the one or more periods of time.

8. The system as recited in claim 7, wherein the one or more characteristics comprises amplitude, power and duration of the identified harmonic or random oscillation for the one or more periods of time.

9. The system as recited in claim 8, further comprising generating a display of one or more relationships between the first amount of harmonic activity and the second amount of random activity identified in the EEG signal based on the one or more characteristics.

10. The system as recited in claim 9, wherein the amount of power in the first amount of harmonic activity and the second amount of random activity are calculated and displayed as a graph of power versus time and power versus an agent concentration.

11. The system as recited in claim 1, further comprising the processing device being configured to separate the EEG signal into frequency ranges comprising a plurality of filtered signal streams.

12. The system as recited in claim 11, further comprising the processing device being configured to identify the one or more harmonic segments having one or more sequences of cycles having substantially identical cycle length in each of the plurality of filtered signal streams.

13. The system as recited in claim 11, further comprising the processing device being configured to generate a filter based on the one or more harmonic segments and the one or more random segments, wherein the processing device divides the signal in a specified frequency range into two outputs.

14. The system as recited in claim 13, wherein the one or more harmonic segments and the one or more random segments are displayed separately on the display device.

15. A method of analyzing EEG signals using an electrode array, a processing device, a display device and a memory, wherein the memory contains executable programming, the method comprising the steps of:
a) processing via the processing device, an EEG signal received from the electrode array, the EEG signal being associated with brain activity of a subject, wherein the processing device identifies one or more random segments and one or more harmonic segments of the EEG signal in one or more frequency bands; and
b) causing to display on the display device by the processing device, on a real-time basis, at least one or both of: a filtered EEG signal from the EEG signal, wherein the filtered EEG signal comprises a display of the EEG signal with the one or more random and harmonic segments of the EEG signal removed; and a modified EEG signal, wherein the modified signal includes a display of the EEG signal with the one or more random and one or more harmonic segments visually identified.

16. The method according to claim 15, further comprising the steps of:
a) identifying in the EEG signal a first amount of harmonic activity identified in the EEG signal and a second amount of random activity identified in the EEG signal in at least one frequency band of the plurality of frequency bands, the first amount of harmonic activity and the second amount of random activity being identified over one or more periods of time; and
b) generating for display on the display device one or more characteristics of the EEG signal for each of the one or more periods of time.

17. The method according to claim 16, further comprising the step of generating a display of one or more relationships between the first amount of harmonic activity and the second amount of random activity identified in the EEG signal based on the one or more characteristics.

18. The method according to claim 17, further comprising the step of identifying the one or more harmonic segments having one or more sequences of cycles having substantially identical cycle length in each of the plurality of filtered signal streams.

19. The method according to claim 18, further comprising the step of generating a filter based on the one or more harmonic segments and the one or more random segments, wherein the processing device divides the signal in a specified frequency range into two outputs.

20. The method according to claim 19, further comprising the step of displaying the one or more harmonic segments and the one or more random segments on the display device.
